(19) United States
(12) Patent Application Publication
(10) Pub. No.: US 2005/0260569 A1
(43) Pub. Date: Nov. 24, 2005

(54) Method, device and system for detecting the presence of microorganisms

(75) Inventors: Michel Houde, Coteau-du-Lac (CA); Michel Grenier, St-Hyacinthe (CA)

Correspondence Address: MAYER, BROWN, ROWE & MAW LLP, 1909 K STREET, N.W., WASHINGTON, DC, 20006, US

(73) Assignees: Biophage Inc., Montreal (CA); NSW Controle Inc., Rock Forest (CA)

(21) Appl. No.: 11/190,936

(22) Filed: Jul. 28, 2005

Related U.S. Application Data
(63) Continuation of application No. 10/184,723, filed on Jun. 28, 2002, now abandoned.

(51) Int. Cl. C12Q 1/70; G01N 33/554; G01N 33/569; C12M 1/34

(52) U.S. Cl. 435/5; 435/287.2; 435/7.32

(57) ABSTRACT

A method, device and system for detecting the presence of microorganisms in an air sample taken from either a gaseous or liquid environment are described. The method including the steps of a) capturing with a filter microorganisms from the air sample; b) recovering from the filter with a liquid at least some microorganisms captured by the filter in step a); and c) detecting the presence of the at least some microorganisms in the liquid. The device and system include a chamber through which the air sample to be examined is circulated; a filter for capturing microorganisms susceptible to be present in the air sample; a liquid cooperating with the filter for recovering captured microorganisms therefrom; and a detector for detecting the presence of microorganisms in the liquid. The method, device and system are particularly useful for the continuous and on-site control, monitoring and detection of microorganisms, such as bacteria, parasites, fungi, viruses and the like, which may be present in an air sample, taken from an air duct of a ventilation system.

### CROSS REFERENCE TO RELATED APPLICATION

This application is a continuation of U.S. Ser. No. 10/184,723, filed Jun. 28, 2002, which is incorporated, in its entirety, herein by reference.

### FIELD OF THE INVENTION

The present invention relates to a method, device and system for trapping and detecting the presence of microorganisms in an air sample, taken from either a gaseous or a liquid environment. More particularly, the present invention relates to a method, device and system which are used for the continuous on-site monitoring and detection of microorganisms, such as bacteria, parasites, molds, fungi, viruses and the like, which may be present in an air sample, taken from either a gaseous or a liquid environment to be examined, whether the environment be indoors or outdoors. The present invention may be used for a wide range of applications and purposes.

### BACKGROUND OF THE INVENTION

It is well known in the art that the microorganism contamination of an indoor environment, such as the air and water systems thereof, can generate serious health problems. Indeed, high humidity, reduced ventilation, tighter buildings, and HVAC (Heat Ventilation and Air Conditioning) systems that contain water or produce condensation (humidifiers, cooling coils, etc) allow for the growth and propagation of various microorganisms. The proliferation of antibiotic-resistant strains, such as the Vancomycin-Resistant Enterococcus (VRE) and Methycillin-Resistant Staphylococcus aureus (MRSA) bacteria, has contributed to aggravate the aforementioned type of health problem.

It is also known in the art that certain environments where most susceptible people are present, such as intensive care units (ICUs), burn units, isolation rooms, nurseries, schools and residential homes, have shown to be potential reservoirs of pathogens and thus constitute a potential hazard to their residents. Even healthy adults are increasingly exposed to different contaminated indoor conditions, such as the sick-building syndrome for example, which is caused by fungi colonizing air filters and air ducts. Also, infectious microorganisms can contaminate the air in hospitals, research labs, food processing facilities, and public buildings, as well as confined spaces, such as aircrafts and subway systems. Furthermore, infectious microorganisms can also contaminate the water systems of orchards, vineyards and other high-managed agricultural areas. Similarly, public and private water supplies have been shown to frequently host pathogens such as Escherichia coli (E. coli) O157:H7, Legionella pneumophila (L. pneumophila) and Cryptosporidium parvum (C. parvum). Consequently, air or liquid sampling is often undertaken in order to diagnose and control microorganism air/liquid contamination, as well as a means of quantitatively and qualitatively monitoring such contamination.

Known in the art are numerous methods for detecting microorganisms in gaseous and liquid environments (Balows, A, 1991, Manual of Clinical Microbiology, 5^{th} Edition; Balows, A, Hausler W J, et al, eds, American Society for Microbiology, pub; 1217 pages), of which a few major approaches are briefly discussed hereinbelow. The first one essentially consists in amplifying the number of pathogens present in a sample taken, by growing the microorganism(s) present in the sample in an appropriate culture medium. For example, agar plates containing different media are inserted in an Andersen sampler and microorganisms present in the air are then sucked into the sampler impact and trapped into a suitable culture plate. After an appropriate incubation period, colonies grown on the plate are used for identification and quantification of microorganisms in the air. The second approach essentially consists in the immunodetection of specific constituents of the microorganism (e.g. ELISA, immunochromatographic tests, biosensors, etc). More recently, a third approach has emerged and is becoming more and more popular, which essentially consists in the amplification of nucleic acids of the genome of the microorganism by polymerase chain reaction (PCR) techniques and the subsequent identification of the amplified material (DNA testing technology).

However, a major problem associated with the above-mentioned techniques and with most of the other techniques known in the art is that they are all used to detect microorganisms in samples collected on a punctual basis, i.e. at different time points. Indeed, the general procedure for identifying a specific microorganism in a gaseous or a liquid environment is usually as follows: (a) identification of a need for a microbiological analysis; (b) manual installation of a collection sampler; (c) operation of the collection sampler for a given period of time (usually one to sixty (60) minutes); (d) transfer of the sample to a detection unit; and (e) generation of a detection signal. For example, to ensure that the air of a neonatal intensive care unit is free of the presence of deadly pathogens such as E. coli O157:H7, Klebsiella pneumoniae (K. pneumoniae) and Staphylococcus aureus (S. aureus), one must typically collect air samples at different locations of the building and at given time intervals (Chandrasekar et al., 1997, Indian Medical Association 95(3):72-77). In terms of costs, time and human resources required, this type of sampling is often more expensive than the testing procedure itself. In addition, there is a high risk that the person assigned to perform this task will be exposed to hazardous pathogen(s) which may be present in the environment to be sampled. With regards to these safety and cost-efficiency considerations, it is actually impractical and most oftenly impossible to insure a daily, weekly and/or even monthly monitoring of the cleanliness of the air in a building or in a water system.

Therefore, in view of the above, it would be useful to have a device and corresponding integrated system which could provide a fully automated processing of all the above-mentioned steps in a continuous manner, in a way similar to that of a smoke detector, thereby allowing a user to install such a device before a microbiological problem arises, the device being able to run for extended periods of time, such as several weeks or months, in order to ensure a continuous and on-site monitoring of the presence of microorganism(s). Furthermore, it would also be useful to provide a device and/or method for the continuous and on-site detection of microorganisms which could be used either for a gaseous or liquid environment, through extended periods of time, such as several days, weeks or even months. Indeed, such a detection or monitoring of microorganisms would be helpful for the control of microorganisms in the air of “clean” areas (e.g. intensive care units, white rooms, etc.); in ventilation ducts of healthcare settings, schools, convention theaters, and residential homes; and/or in water systems of public water companies for example. Orchards, vineyards and other high-managed agricultural areas could also benefit from such integrated device/system.

Known to the applicant are the following U.S. patents which describe various methods and devices for detecting microorganisms.

U.S. Pat. No. 4,200,493 discloses a method for detecting and enumerating microorganisms in a growth medium, based on the change of potential between two electrodes arising by the migration and accumulation of microorganisms. However, no collection method seems to be described and the samples have to be manually added to the apparatus. In addition, the method described for the detection of microorganisms is not specific as it doesn't allow the discrimination between dead and live bacteria present in the air, nor can it discriminate between pathogenic and nonpathogenic bacteria.

U.S. Pat. No. 6,101,886 discloses a method for air sampling based on the properties of aerogel, enabling the collection, separation and concentration of aerosols. However, this device is not designed for the continuous monitoring of ambient air, but rather is devised for operating during only a predetermined period of time. Furthermore, this device does not seem to provide an automated process, as a manual operation is required for the introduction of new filters onto the carrier. Finally, no detection assay is included in the device, a fluid sample requiring to be conveyed to another analytical apparatus.

U.S. Pat. No. 6,143,555 discloses a method for the detection, identification and measurement of microorganisms based on the dissolution of oxygen caused by the respiration of the microorganisms, either in the presence or in the absence of antibiotics. No combination of sampling and testing seems to be described in this patent, as the test sample has to be injected manually into the apparatus. Also, this method only provides a gross characterization of the microorganisms, based on culture medium selectivity and antibiotics resistance. Moreover, the identification profile of the species present in the sample taken is not as formal as the one obtained using specific ligands, such as phages and antibodies.

U.S. Pat. No. 6,192,767 discloses a method for collecting airborne particles such as bacteria, mycetes, spores and viruses. This device is essentially composed of a sampling chamber, an aspirative turbine and a power supply. The sampling chamber is cylindrical and includes an adhesive tape on which are captured the particles. This device does not provide a continuous monitoring of ambient air, nor an automation thereof, as a manual operation is required for the removal of the sampling chamber. Furthermore, no detection assay seems to be included in the device, the sampling chamber necessitating to be removed from the apparatus, closed in a sterile case and sent to a laboratory for analysis.

U.S. Pat. No. 6,244,096 discloses a method for the detection of microorganisms based on the detection by a sensor of a marker gas released by the microorganism. Similarly to the above-mentioned patents, no system for the continuous and on-site sampling of microorganisms seems to be described. Also, this method only provides a gross characterization of the microorganisms, based on the profile of gases released by the microorganism.

All the above-discussed patents describe either a method for collecting or a method for detecting microorganisms, but none of them seem to describe a method which integrates both the sampling and the detecting of microorganisms.

U.S. Pat. No. 5,918,259 discloses a method and apparatus for monitoring air for the presence of bacteria and spores. This device uses a high volume virtual impactor to collect the particulate fraction from the air. The particulate fraction is then transferred to a processing fluid. Detergents and luminescent test reagents are then added to the processing fluid line so that measurable amounts of ATP are released from the bacterial cells present in the fluid. The presence of ATP is finally monitored by converting the light signal emitted from the processing line. Although this method claims to provide a continuous and automated monitoring, it does not use a filter and it does not seem to be able to discriminate between dead and live bacteria present in the air, and does not seem to discriminate either between pathogenic and nonpathogenic bacteria, as ATP is a component of all cells.

U.S. patent application No. 2001/0029793 A1 (continuation in part of U.S. Pat. No. 6,267,016) also discloses a combined method. In this case, microorganisms are captured on the arcuate vans of an impact collector as ambient air is aspirated by a fan rotating at a speed superior to 5000 rpm. The arcuate vans are then rinsed and the resulting particulate-laden rinse fluid is then analyzed using an immunoassay-based detection unit. However, this instrument has been designed to run for limited periods of time (“real-time” mode), which is undesirable in some cases.

It is also known in the art that there are several problems and concerns associated with the implementation of continuous and on-site detection of microorganisms. Firstly, the continuous supply of samples to a detection unit is problematic. Secondly, the evaporation of the detection fluid put into contact with an environment such as circulating air is not only possible/probable but very often undesirable. For example, an obvious way to detect bacteria in the air would be to lay open a recipient or tube containing a growth medium selective for that bacterium. However, even a relatively large volume (10-100 mL) of medium would evaporate in just a few days in a circulating air environment, which is very disadvantageous. The evaporation would be even faster with agar plates. Thirdly, the stability of the test reagents in an open environment, such as a ventilating duct or tap water for example, may be short. Most reagents used in the tests typically intended for the detection of microorganisms require to be stored at about 4° C. At room temperature (20-25° C.), these test reagents usually lose a significant amount of their activity and efficiency each day. Fourthly, there would be the need to automatize each step of the collection sampling and of the detection procedure. Highly sophisticated and expensive automated analyzers are commercially available (Mouritsen, 1999, Analytical Chemistry 71(2): 366R-372R; Jungkind, 2001, J Clinical Virology 20 (1-2): 1-6) but cannot be used in the context of a continuous on-site monitoring for practical as well as cost-efficiency reasons. On the other hand, many “rapid” tests have now been recently introduced, particularly for the detection of bacteria (Shiba, 1998, Rinsho Biseibutsho Jinsoku Shindan Kenkyukai, Shi 9(2): 73-81) but these tests are usually less sensitive than the ones previously discussed. Moreover, very few automated methods are available for the collection of samples. Finally, there would be the need to develop technology enabling the transmission of the test result from the test site to the “control board”.

Hence, in view of the above-discussed, there is a substantial need for a safer and more cost-effective method enabling on-site and continuous control, detection and/or monitoring of the presence of microorganisms in an environment, whether gaseous or liquid, than what is possible with the methods and devices known in the prior art.

### SUMMARY OF THE INVENTION

The object of the present invention is to provide a method, device and/or system for detecting the presence of microorganisms in an air sample which satisfies some of the above-mentioned needs and which is thus an improvement over the methods and devices known in the prior art.

Preferably, the present invention is used for an on-site and continuous control, detection and/or monitoring of the presence of microorganisms in an environment, whether the environment be gaseous or liquid.

In accordance with the present invention, the above object is achieved by a method for detecting and/or monitoring the presence of microorganisms in an air sample, the method comprising the steps of:
a) capturing with a filter microorganisms from said air sample;
b) recovering from the filter with a liquid at least some microorganisms captured by the filter in step a); and
c) detecting the presence of said at least some microorganisms in the liquid.

According to another aspect of the invention, there is also provided a device for detecting the presence of microorganisms in an air sample, the device comprising:
a chamber through which the air sample is circulated, the chamber having an inlet for receiving the air sample into the chamber and an outlet for releasing the air sample from the chamber, the air sample circulating through the chamber from the inlet to the outlet thereof;
a filter positioned within the chamber between the inlet and the outlet, the filter allowing the air sample to pass therethrough while capturing microorganisms from said air sample;
liquid receiving means positioned within the chamber for receiving a liquid, the liquid receiving means cooperating with the filter for ensuring a contact of at least one portion of the filter with the liquid and thereby recovering at least some microorganisms captured by the filter; and
detecting means for detecting the presence of at least some of the microorganisms within the liquid.

According to yet another aspect of the invention, there is also provided a system for detecting the presence of microorganisms in an air sample, the system comprising:
a chamber through which the air sample is circulated, the chamber having an inlet for receiving the air sample into the chamber and an outlet for releasing the air sample from the chamber, the air sample circulating through the chamber from the inlet to the outlet thereof;
a filter positioned within the chamber between the inlet and the outlet, the filter allowing the air sample to pass therethrough while capturing microorganisms from said air sample;
a liquid present in the chamber, the liquid operatively cooperating with the filter so as to ensure a contact of at least one portion of the filter with the liquid and a recovery of at least some microorganisms captured by the filter; and
detecting means operatively connected to the liquid for detecting the presence of said at least some microorganisms within the liquid.

The present invention is particularly advantageous in that it may be used for detecting the presence of microorganisms in an air sample which may be taken from different environments, whether gaseous or liquid. The present invention is also advantageous in that it allows for a continuous and on-site control, monitoring and detection of microorganisms, over extended periods of time, such as several weeks or months.

The present invention is also a substantial improvement over the prior art in that it provides a method and corresponding device/system for (a) continuously collecting microorganisms from a gaseous/liquid environment over an extended period of time; (b) continuously transferring the microorganisms to a detection fluid; and (c) continuously measuring the concentration of microorganisms present in the detection fluid.

The device and system according to the present invention may be produced at a very competitive price, is portable, automated, and practically silent during operation, which would be very advantageous over the devices known in the prior art.

Other objects, advantages and other features of the present invention will become more apparent upon reading of the following non-restrictive description of preferred embodiments thereof, given for the purpose of exemplification only with reference to the accompanying drawings.

### BRIEF DESCRIPTION OF THE DRAWINGS

FIG. 1 is a fragmentary schematic perspective view of a system according to a first preferred embodiment of the invention, the system being used for detecting the presence of microorganisms in an air sample taken from a gaseous environment.

FIG. 2 is a fragmentary schematic perspective view of a system according to the first preferred embodiment of the invention, the system being used now for detecting the presence of microorganisms in an air sample taken from a liquid environment.

FIG. 3 is a fragmentary schematic perspective view of a system according to a second preferred embodiment of the invention, the system being used for detecting the presence of microorganisms in an air sample taken from a gaseous environment.

FIG. 4 is a fragmentary schematic perspective view of a system according to the second preferred embodiment of the invention, the system being used now for detecting the present of microorganisms in an air sample taken from a liquid environment.

FIG. 5 is a cross-sectional view of the device according to the first preferred embodiment of the invention.

FIG. 6 is a cross-sectional view of the device according to the second preferred embodiment of the invention.

FIG. 7 is a perspective view of detecting means according to yet another preferred embodiment of the invention.

In the following description, the same numerical references refer to similar elements. The embodiments shown in the accompanying drawings are preferred.

### DETAILED DESCRIPTION OF THE INVENTION

As shown in the accompanying drawings, the present invention relates to a method, device 1 and system for detecting the presence of microorganisms in an air sample, taken from either a gaseous or a liquid environment.

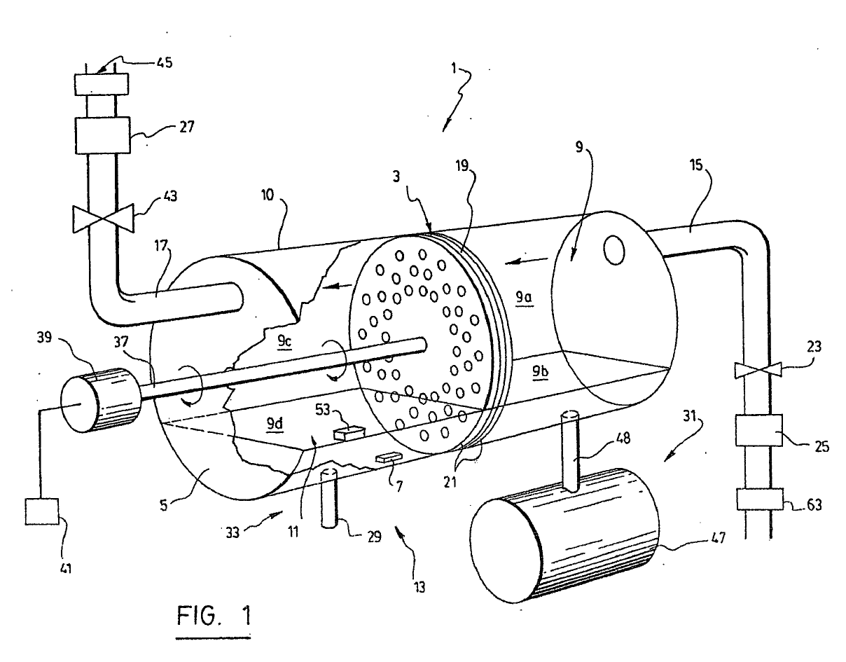

Broadly described, the detecting method according to the present invention is essentially concerned with (a) the trapping or capturing via a filter 3 of microorganisms from an air sample taken from a gaseous and/or liquid environment to be analyzed, such as air and/or water for example; (b) the repeated contact of that filter 3 with an appropriate liquid 5 (“detection fluid”), using preferably either a “rolling circle” or a “fluid dripping” principle (see FIGS. 1-2 and 3-4 respectively); and (c) the specific detection of the microorganisms in the detection fluid 5 by appropriate means.

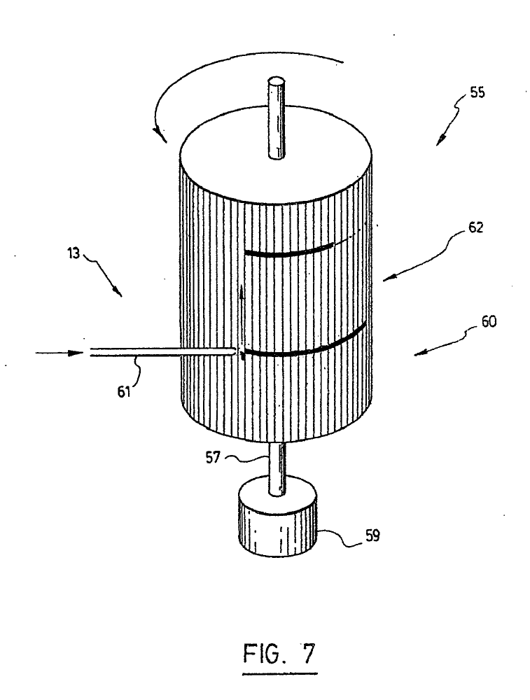

Preferably, the detecting step is carried out with suitable detecting means 13, such as a biosensor 7 for example, which preferably comprises antibodies for specifically recognizing the at least some microorganisms; but the detecting step may also be carried out with a biosensor 7 comprising antibodies for specifically recognizing bacteriophages capable of replicating in the at least some microorganisms, in which case the recovering step is preferably carried out with a liquid 5 comprising a culture medium containing bacteriophages (phages). The detecting step may also be carried out in other suitable manners, with other suitable detecting means 13, as apparent to a person skilled in the art. Indeed, in the absence of a biosensor 7, a sample of liquid 5 recovered in step b) may be taken and analyzed for the presence of microorganisms by other appropriate detecting means 13 known in the art, such as with immunochromatographic procedures for example, as illustrated in FIG. 7. Thus, any other suitable detecting means 13 capable of detecting directly or indirectly the presence of the at least some microorganisms captured by the filter 3 from the sample being examined, can be used for the detecting step, as apparent to a person skilled in the art. Biosensors 7, immunochromatographic procedures and individual sampling are just a few examples of detecting means 13 which may be used according to the present invention.

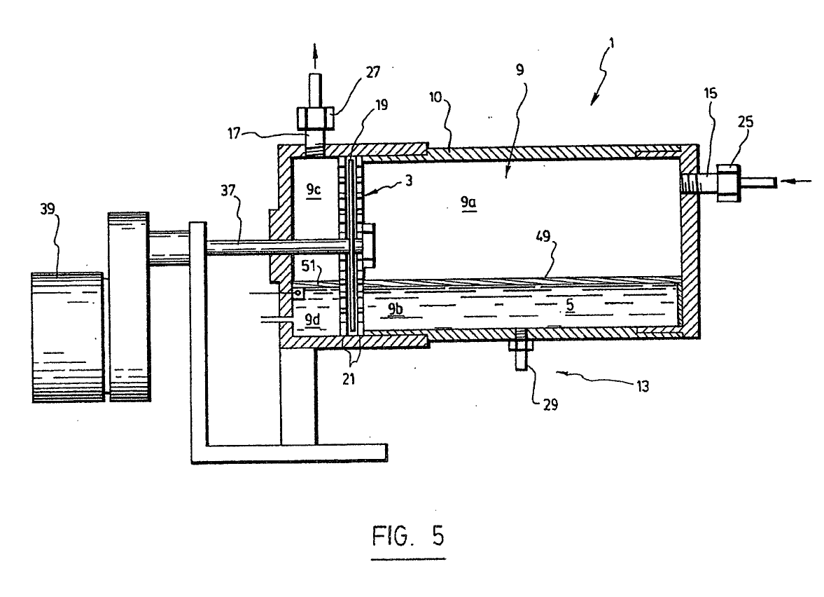

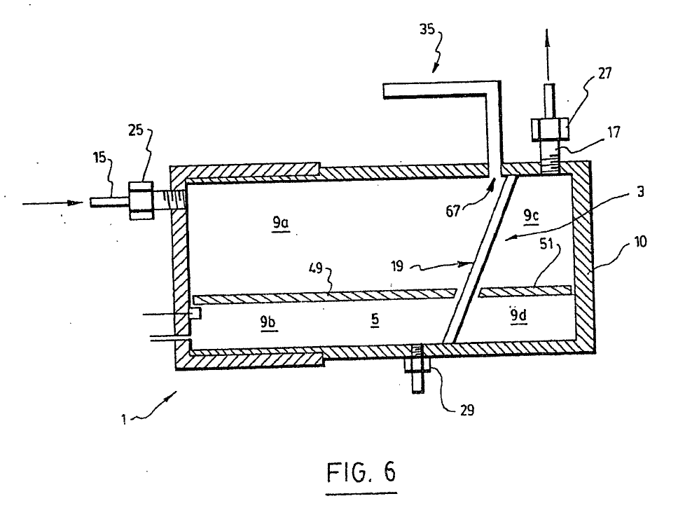

According to the present invention, the method is preferably carried out with a device 1 as described herein. As shown in the accompanying figures, the device 1 comprises a chamber 9, a filter 3, liquid receiving means 11, and detecting means 13. The air sample to be examined is circulated through the chamber 9 and, as better shown in FIGS. 5 and 6, the chamber 9 has an inlet 15 for receiving the air sample into the chamber 9 and an outlet 17 for releasing the air sample from the chamber 9, the air sample circulating through the chamber 9 from the inlet 15 to the outlet 17 thereof. As also shown, the filter 3 is positioned within the chamber 9 between the inlet 15 and the outlet 17, the filter 3 allowing the air sample to pass therethrough while capturing microorganisms from it. The liquid receiving means 11 are positioned within the chamber 9 for receiving a liquid 5 (previously referred to also as the “detection fluid”). The liquid receiving means cooperate with the filter 3 for ensuring a contact of at least one portion of the filter 3 with the liquid 5 and thereby a recovery of at least some microorganisms captured by the filter 3. The detecting means 13 are used for detecting the presence of the at least some microorganisms within the liquid 5 which have been trapped by the filter 3.

According the first preferred embodiment of the invention, as better shown in FIGS. 1, 2, and 5, the filter 3 consists of an assembly having a filtering membrane 19 positioned between a pair of perforated holders 21, the assembly being rotatable within the chamber 9 and the filtering membrane 19 having a surface in contact with the liquid 5 so that microorganisms captured by the filtering membrane 19 can be released into the liquid bath of the chamber via rotation of the assembly.

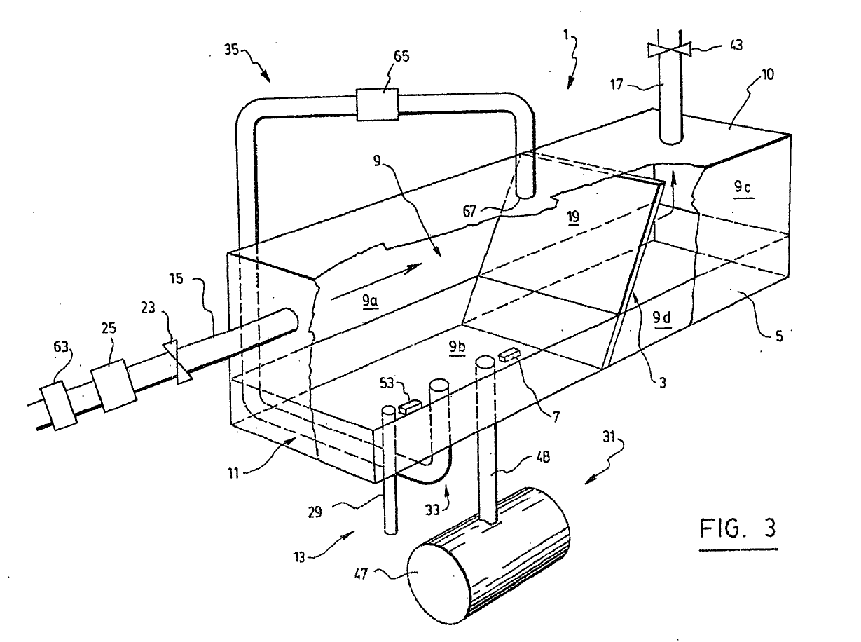

According the second preferred embodiment of the invention, as better shown in FIGS. 3, 4, and 6, the filter 3 comprises a filtering membrane 19, a portion of the liquid 5 in the chamber 9 flowing from a top portion of the membrane 19 toward a bottom portion thereof so that the microorganisms captured by the filter 3 can be “washed” down into the bottom portion of the chamber 9, wherein detection is preferably carried out.

In either case, the inlet 15 of the chamber 9 may be provided with a valve 23 for selectively controlling an amount of air sample entering the chamber 9 and/or with a pre-filter 25 for filtering substances having a size larger than the microorganisms from the air sample prior to entering the inlet 15, as shown in FIGS. 1-4.

Similarly, the outlet 17 of the chamber 9 may be provided with a post-filter 27 for filtering the air sample released from the chamber 9 and/or the chamber 9 may comprise a liquid outlet valve 29 for allowing collection of a sample of the liquid 5 contained in the chamber 9, as also shown in FIGS. 1-4.

According to the present invention, the aforementioned device 1 is used within a system for detecting the presence of microorganisms in an air sample, taken from either a gaseous, liquid and/or ultimately even a particulate environment to be examined, as apparent to a person skilled in the art. As better shown in FIGS. 1-6, the system comprises a chamber 9, a filter 3, a liquid 5 present in the chamber 9, and detecting means 13. As aforementioned, the air sample is circulated through the chamber 9 which has an inlet 15 for receiving the air sample into the chamber 9 and an outlet 17 for releasing the air sample from the chamber 9, the air sample circulating through the chamber 9 from the inlet 15 to the outlet 17 thereof. The filter 3 is positioned within the chamber 9 between the inlet 15 and the outlet 17, the filter 3 allowing the air sample to pass therethrough while capturing microorganisms from the air sample. The liquid 5 present in the chamber 9 operatively cooperates with the filter 3 so as to ensure a contact of at least one portion of the filter 3 with the liquid 5 and a recovery of at least some microorganisms captured by the filter 3. The detecting means are operatively connected to the liquid 5 having interacted with the filter 3 for detecting the presence of said at least some microorganisms within the liquid 5.

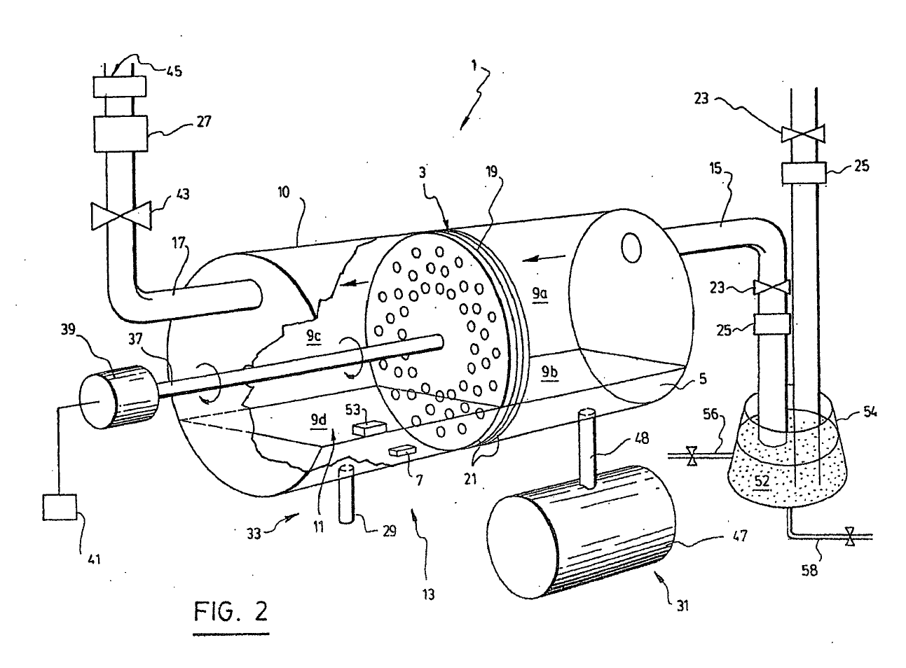

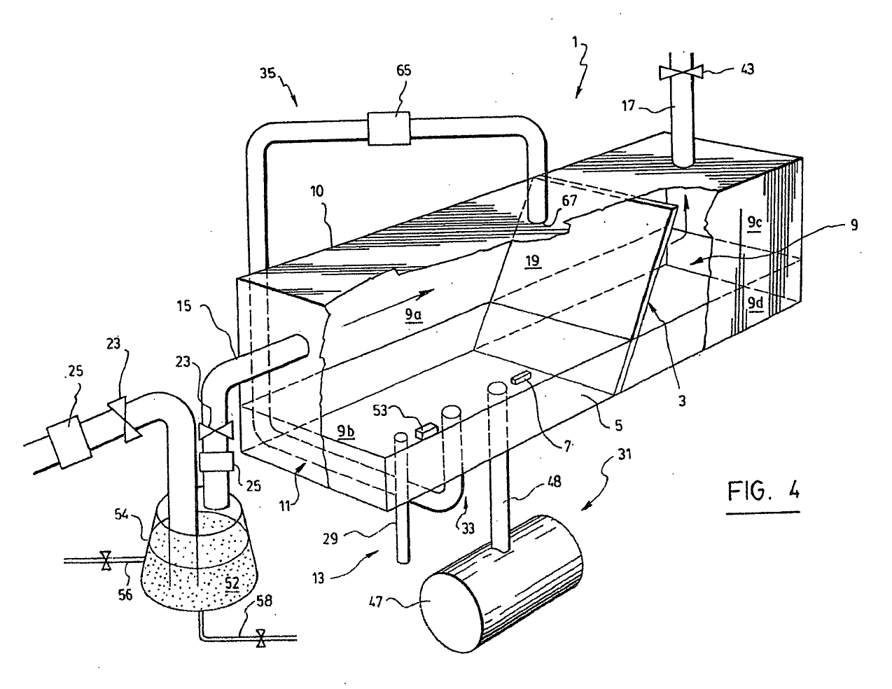

The air sample to be examined may consist of a continuous flow of air for example, as shown in FIGS. 1 and 3, or may consist of an air mixture effervesced from a liquid mixture, as better shown in FIGS. 2 and 4, thereby enabling the present invention to be used in both gaseous and/or liquid environments, as will be explained in greater detail hereinbelow. The liquid mixture typically consists of a liquid sample susceptible to contain microorganisms, and may be a water sample taken from a water supply to be examined for example.

The liquid 5 or “detection fluid” in the chamber 9 may comprise a culture medium for promoting proliferation of said at least some microorganisms so as to be more easily detected by the detecting means 13 of the system, but the liquid 5 may alternatively comprise a culture medium containing bacteriophages, depending if the detecting means are intended for specifically recognizing bacteriophages capable of replicating in the captured at least some microorganisms which are transmitted to the liquid 5 via its interaction with the filter 3.

The system preferably comprises regularizing means for maintaining a predetermined level of liquid 5 in the chamber 9. Preferably, the regularizing means comprise feeding means 31 for feeding liquid 5 into the chamber 9 and draining means 33 for draining liquid 5 from the chamber 9, the draining means 33 being preferably connected to recirculating means 35 for recirculating drained liquid back into the chamber, particularly in the case of the second preferred embodiment, as better shown in FIGS. 3 and 4.

According to the present invention, the system and the components thereof are preferably devised to detect microorganisms such as bacteria (e.g. E. coli 0157:H7, Legionella pneumophila, Bacillus anthracis, Neisseria miningitidis, Mycobacterium tuberculosis), fungi (e.g. Aspergillus flavus, fumigatus, Niger, Histoplagma capsulatum, Coccidioides imitis), parasites (e.g. protozoa, Giardia lamblia, Cryptosporidium parvum), viruses (e.g. small pox, influenza virus, rubella virus) and the like.

### Detailed Description of a First Specific Preferred Embodiment of the Invention

A first embodiment of the present invention, also known as the “rolling circle” embodiment, is illustrated in FIGS. 1, 2, and 5. These figures show an example of the device 1 acting as an air-monitoring unit. The unit shown in FIGS. 1 and 5 is particularly useful for detecting an exposure to harmful microorganisms that might be present in the air environment of facilities such as hospitals, schools, industries, houses, farms and the like. In operation, the air-monitoring unit is preferably operatively connected in a continuous manner to an air duct of the ventilation system of the corresponding facility to be inspected.

The unit shown in FIG. 2 is particularly useful for detecting microorganisms in a liquid sample.

The air-monitoring unit preferably includes a pre-filter 25, an air inlet 15, an inlet valve 23, a housing 10, a rotating drive shaft 37, a motor 39, a power supply 41, an outlet valve 43, a post-filter 27, an air pump 45, an air outlet 17, a fluid container 47, a connecting line 48 between the fluid container 47 and the housing 10, a biosensor 7, a liquid outlet 29, a control board (not shown), relay wires from the different components of the system to the control board, and supports for the housing 10, the motor 39 and other components of the system.

As better shown in FIGS. 1 and 2, the air coming from the pre-filter 25 is directed through an air inlet 15, which is connected to a screwable cover that can be hermetically fixed to the inlet portion of the housing 10. The inlet portion of the housing 10 is preferably separated into two upper and lower chambers 9a, 9b by a first separator plate 49 (not shown in FIGS. 1-2 for simplicity). The upper chamber 9a is preferably empty, allowing the free circulation of air coming from the air inlet 15 while the lower chamber 9b is preferably filled with the liquid 5 or “detection fluid”. The outlet portion of the housing 10 is designed to be tightly screwed into the inlet portion of the housing 10 and to receive an assembly formed of a filtering membrane 19 and two filter holders 21. These three parts each bear a hole in their center allowing them to be serially installed on the rotating shaft 37. Appropriate fastener(s) allow the filtering membrane 19 and filter holders 21 to be fixed onto the shaft 37 and to rotate therewith. A second separator plate 51 (also not shown in FIGS. 1-2 for simplicity) is preferably installed just below the rotating shaft 37, at the same level as the first separator plate 49 of the inlet portion of the housing 10 and similarly, divides the outlet portion of the housing 10 into an upper chamber 9c, which is preferably empty, allowing the free circulation of air coming from the filter assembly, and a lower chamber 9d which is preferably filled with the detection fluid 5. The air outlet 17 is preferably installed on the upper chamber 9c of the outlet portion of the housing 10. When the outlet portion of the housing 10 is screwed into the inlet portion of the housing 10, the two separators 49, 51 cover almost all of the surface of lower portion of housing 10, below the drive shaft 37, except for a preferred 1-2 mm wide slot occupied by the filtering membrane 19 and the filter holders 21, therefore greatly reducing the spontaneous evaporation of the detection fluid 5 contained in the lower chambers 9 of the housing 10. The rotating shaft 37 allows the filtering membrane 19 to be dipped into the detection fluid 5 contained in the lower chambers 9b, 9d. A volume sensor 53 is preferably installed in the lower chamber 9b and is connected by a relay wire to the control board of the system. Preferably also, signals provided by the volume sensor 53 to the control board allows the volume to be kept at a constant level, as the control board is preferably further connected to a liquid feeding pump by another relay wire. The pump draws detection fluid from the container 47 and into the lower chamber 9b via line 48. Preferably also, a detection biosensor 7 is also installed in the outlet lower chamber 9d for the detection of microorganisms and is connected by an appropriate relay wire to the control board of the system.

The air-monitoring unit typically functions as follows: the pump 45 provides a particular aspiration flow rate ranging preferably from about 10 to 200 liters per minute (L/min) into the air inlet 15 and then, into the upper chamber 9a. It is worth mentioning that other suitable flow rates may be used with the present invention, as apparent to a person skilled in the art. Alternatively, air pump 45 can be replaced by a blower 63 installed upstream of the air inlet 15, as illustrated in FIG. 1. It is to be noted that the blower 63 and the pump 45 are not mutually exclusive and can be both installed on the same instrument in order to ensure a greater airflow. Also, as previously mentioned and as better illustrated in FIG. 2, the air sample to be examined may originate from a liquid environment 52, in which case it preferably consists of a sample of air which has been nebulized from a liquid sample containing or susceptible to contain microorganisms. This liquid sample may consist of an isolated liquid sample contained in a nebulizer 54 operatively connected to the air inlet 15 of the system, as better shown in FIG. 2, or may consist of a liquid sample taken from a continuous liquid stream circulating via the nebulizer 54 by means of suitable liquid inlet(s) 56 and outlet(s) 58 being connected to the nebulizer 54, as also shown in FIG. 2. These inlets/outlets 56, 58 may also be provided with suitable filters, valves, etc., depending on the applications for which the system is intended and the type of liquid environment to be examined. It is worth mentioning however, as apparent to a person skilled in the art, that other suitable means may be used for taking a liquid sample to be examined, and converting it to a substantially gaseous form so that the sample may be used with the device 1 according to the present invention. Hence, in view of the above, it can be easily understood that the present invention may be used for carrying out all sorts of liquid contamination detections.

In either case, whether the air sample to be examined comes from a gaseous environment or a liquid environment 52 containing or susceptible to contain microorganisms, it is directed to the chamber 9 of the device 1 by means of the air inlet 15, as shown in FIGS. 1 and 2. Preferably, the air inlet 15 is equipped with a conical nozzle (not shown); a pre-filter 25 of opening sizes typically 4 micrometers (μm) to 6 μm wide for separating, ambient dust and flora for example, from the incoming air; a valve 23; and a security device (not shown) which prevents the exit of microorganisms from the housing 10 in the absence of a negative gaseous pressure (from the “outside” to the “inside”). Because of the aspiration flow, microorganisms present in the air and entering into the upper chamber 9a are driven directly onto the filter assembly. As aforementioned, the filter membrane 19 is preferably held in place by a pair of filter holders 21, which both contain sufficient inner empty spaces to allow the microorganisms to enter in contact with the filter membrane 19 and the air to pass through it. Filters such as the Durapore™ Membrane 0.22 μm GV (Millipore, Bedford, Mass., USA) for example can used. Because of the rotation of the shaft 37 onto which the filter membrane 19 is fixed, the microorganisms present on the filter membrane 19 are periodically transferred to the lower chamber 9 where they are detached from the filter 3 by the detection fluid present in the chamber 9. The filter assembly can rotate either in a clockwise or a counterclockwise direction, typically at a speed of about 6-10 rpm. The optimal speed depends on parameters such as the diameter of the filter membrane 19, the humidity of the air and the temperature of the air, and is preferably selected to achieve a constant moistening of the filter 3, the best preservation of the microorganisms, the minimal evaporation of the detection fluid 5, and the lowest consumption of energy possible.

The detection fluid 5 is preferably a culture medium favoring the growth or the maintenance of the microorganism to be detected (e.g. LB medium for Escherichia coli bacteria). However, it can also be any kind of liquid 5 allowing the detection of a marker specific to the microorganism to be detected. In order to improve the specificity of the detection fluid 5, to reduce the degradation of the test reagents potentially caused by heterologous microorganisms and to increase the stability of the system, several agents such as fungicides, virucidal agents and selective antibiotics can be added to the fluid. For example, a fungicide such as amphotericin B (Fungizone™, Gibco BRL-Life Technologies, Rockville, Md., USA) can be added to the detection fluid to eliminate viable fungi entering into the detection chamber 9 or produced by the development of spores brought by air. To the same extent, an antibiotic specific to gram-positive bacteria, such as erythromycin (Abbott, Chicago, Ill., USA), can be added when the detection of gram-negative bacteria such as E. coli O157:H7 is required.

The level of fluid 5 in the lower chambers 9b, 9d is preferably kept at a constant level by using a volume sensor 53 for example, a control board, a liquid pump and a fluid container 47, which supplies the detection fluid 5 to the lower chamber 9b through a connecting line 48. The volume sensor 53 is preferably a sensitive piezoelectric crystal, such as the ones manufactured and distributed by Universal Sensors (Metairie, La., USA) for example, but it could be a much more simpler device such as a float for example. After passing through the filter 3, the air is exhausted through an air outlet 17 equipped preferably with a HEPA filter or any similar kind of device which can prevent the passage of viruses and other microorganisms (typically, particles of diameter superior or equal to 10 nm).

Preferably, the motor 39 and pumps of the air monitoring unit are energized with an AC/DC power supply 41 so as to ensure its use for an extended period of time, e.g. several months. However, in the case where the present system has to be installed in an environment devoid of any AC/DC power supply, battery power supplies can also be used for feeding the motor 39 and pumps of the present system since these are generally low-consuming devices.

Preferably, bacteriophages (or phages) specific for the bacterium to be detected are added in the detection fluid 5. It is known that phages will replicate only in the presence of their specific bacterial host, a process which typically leads to a 1000 to 1000000-fold increase in their number in the detection fluid 5 in a 24 hour period. That increase in the phage concentration is detected by the detecting means 13 of the present invention, preferably a biosensor 7 which then transfers a signal by a connecting relay wire to the control board of the system. The biosensor signal is then converted into a detection signal (light, buzzer, electronic message, etc.) informing of the presence of the microorganism in the air sampled. For example, a quartz piezoelectric crystal, such as those manufactured and distributed by Universal Sensors (Metairie, La., USA) for example, can be coated with an antibody specific to the phage present in the fluid. The binding of the phage to the antibody will change the frequence of vibration of the crystal, a signal which can be converted by the control board, using an appropriate interface. As apparent to a person skilled in the art, antibodies that can directly bind to the bacterium (or any other microorganism) can also be used according to the present invention. To the same extent, fragments of DNA, RNA or any derivative of nucleic acid (such as PNA (Peptide Nucleic Acids), for example) deduced from the sequence of the microorganism(s) to be detected may also be used for the production of a specific signal. Other means of detection, such as fluorescence, chemiluminescence, potentiometry, spectrophotometry or immunochromatography, can also be used to generate a detectable signal of the antibody-antigen binding, as also apparent to a person skilled in the art.

Alternatively, a fluid outlet valve 29 may be installed on the lower chamber 9b, allowing the collection of fluid samples at different times after sampling. The fluid sample can then be tested for the presence of microorganisms in an outside location, using microbiological, immunological, PCR-related methods or any other suitable detecting means.

The detecting means 13 may also comprise an immunochromatographic detector in order to improve the stability of the device. Preferably, the immunochromatographic detector comprises an automatic carrousel 55 or conveyer, as better shown in FIG. 7, that can be connected to the fluid outlet valve 29 of the lower chamber 9d.

A membrane sheet is fixed onto the carrousel 55 so as to make a cylinder having for example a diameter of 5 cm and a height of 5 cm. Membranes used for immunochromatographic or lateral flow procedures, such as UniStart™ nitrocellulose membranes (Sartorius A G, Goettingen, Germany) can be used. As better shown in FIG. 7, the carrousel 55 is connected by a shaft 57 to a motor 59, in a principle similar to the one described for the filter assembly shown in FIGS. 1 and 2. The cylinder is rotated in a discontinuous way (typically 6° once a day) so that a complete rotation is done in 60 days. Depending on the stability of the system, stepwise rotations of 3, 2 or 1° can be done if periods of 120, 180 or 260 days are required. The frequency of rotation can also be done on a daily, weekly or even monthly basis. After each rotation, a minimal volume of detection fluid 5 (typically 0.1 mL) is transferred by a liquid dispenser 61 from the lower chamber 9d of the outlet portion of the housing 10 to the basis of the cylinder, in a region called the “conjugate zone” 60. The device ensuring the daily (or weekly or monthly or other) sampling of the detection fluid 5 can be directly connected to the fluid outlet valve. The conjugate zone 60 consists in an anti-phage antibody (Ab # B) conjugated to a label such as colloidal gold. This conjugate is simply deposited and dried in the “conjugate region” of the membrane. When the detection fluid 5 comes into contact with the membrane at the level of the conjugate zone, the conjugate is resuspended. As can be easily understood, the immunocomplexes consisting of phages and antibody-colloidal gold are now free to migrate to the top of the cylinder by capillarity. In order to minimize the lateral dispersion of liquid migrating by capillarity, empty spaces can be inserted between each “strip”, as better shown in FIG. 7. At an appropriate distance above the conjugate zone 60 (typically 2 cm), a second anti-phage antibody (#B) is absorbed onto the cylindrical membrane, creating a “reaction zone” 62. While migrating to the top of the strip, the immunocomplex is captured by the Ab # 2 of the reaction zone 62 and a visible band appears on the cylinder membrane.

A skilled technician familiar with immunochromatographic procedures will find various other arrangements of phages and phage ligands that can lead to a visible signal. To the same extent, a skilled technician familiar with diagnostic systems will easily find other ways to resuspend the phage-ligand(s) complex and to yield a detectable signal. To the same extent, a skilled technician familiar with immunochromatographic procedures will find various arrangements of antibodies and microorganisms that can lead to a visible signal.

Preferably, the monitoring unit is devised to remain functional for several weeks, based on the design of the housing which greatly reduces the evaporation of the detection fluid 5, the stability of all the components selected for the fabrication of the unit and the low-energy features of the motor and/or pumps.

Preferably also, housing 10, separators 49, 51 and fluid container 47 are fabricated from a polymeric material. It is anticipated that injection molded components of suitable quality can be inexpensively produced in large quantities. Preferably, drive shaft 37 is also fabricated from a polymeric material that exhibits good self lubricating properties so that neither bearings nor additional lubricants are required to enable the filtering membrane 19 and the filter holders 21 to freely rotate between the upper and lower chambers 9a-9d. Other suitable materials may be used for the components of the present invention, as apparent to a person skilled in the art.

To ensure the stability of the unit, it is preferred that volumes of detection fluid 5 ranging from 10 to 50 mL be employed. It should be understood that although current methods allow the detection of microorganisms in very small volumes (e.g. microliters) of fluid sample, no system allowing the entering and exiting of a gaseous fluid can totally prevent the evaporation of its detection fluid thereto. As the present invention is intended to be functional for several months, the volume of detection fluid 5 to be used should take account of that evaporation rate, as apparent to a person skilled in the art. It is anticipated that progress in the fields of microfluidics and nanotechnologies will eventually allow the reduction of the volumes required.

### Detailed Description of a Second Specific Preferred Embodiment of the Invention

A second embodiment of the present invention, also known as the “fluid dripping” embodiment, is illustrated in FIGS. 3, 4 and 6. These figures show an example of the device 1 acting as an air-monitoring unit, the second embodiment having a working principle very similar to that of the first embodiment. The unit shown in FIGS. 3 and 6 is particularly useful for detecting microorganisms in a gaseous environment, while the unit shown in FIG. 4 is particularly useful for detecting microorganisms in a liquid environment 52.

The air-monitoring unit according to the second preferred embodiment of the invention includes a pre-filter 25, a housing 10, a power supply (not shown), a blower 63, a fluid pump 65, lines connecting the fluid pump 65 to the housing 10, a fluid container 47, an air outlet 17, a connecting line 48 between the container 47 and the housing 10, a biosensor 7, a volume sensor 53, a liquid outlet 29, relay wires to be connected to a control board (not shown) of the system, and supports for the housing 10 and other parts of the system.

Cross-sectional views of the housing 10 according to the first and second preferred embodiments of the invention are shown in FIGS. 5 and 6 respectively. The only substantial difference between the second embodiment and the first embodiment is that the filtering membrane 19 which is fixed to a single filter holder is not installed on a rotating shaft, but is positioned preferably at an angle. In the first embodiment, as shown in FIG. 1, the filtering membrane 19 is brought to the detection fluid 5 by the rotating shaft 37; whereas in the second embodiment, as shown in FIGS. 3, 4 and 6, it is the detection fluid 5 which is brought to the filtering membrane 19, using the fluid pump 65 which recirculates the detection fluid 5 present in the lower chamber 9b using the connecting lines. The filter 3 according to the second preferred embodiment of the invention may take on other suitable shapes and positions, as well as other cooperations with the housing, as apparent to a person skilled in the art.

The air-monitoring unit according to the second preferred embodiment functions as follows: the air is collected by a pump 45 and directed into the upper chamber 9a. Because of the airflow, microorganisms present in the air are driven directly onto the filter 3. Using the connecting line, the fluid pump 65 pumps the detection fluid 5 from the lower chamber 9b and brings it to a gutter 67 by the corresponding connecting line. The detection fluid 5 drips from the gutter 67 onto the filtering membrane 19, which creates a gravitational descending flow of detection fluid from the gutter 67 to the lower chamber 9b. Because of the gravitational flow, the microorganisms present on the filter 3 are detached from the filtering membrane 19 and collected in a lower portion of the chamber 9. The microorganism is detected by appropriate detecting means 13 in a manner similar to that described for the first embodiment.

Similarly to the previously discussed in reference to FIG. 2, the unit according to the second preferred embodiment may also be provided with a nebulizer 54 and corresponding components (inlet, outlet, etc.) for detecting the presence of microorganisms in a liquid environment 52 to be examined.

### EXAMPLES

The following examples are illustrative of the wide range of applicability of the present invention and is not intended to limit its scope. Modifications and variations can be made therein without departing from the spirit and scope of the invention. Although any method and material similar or equivalent to those described herein can be used in the practice for testing of the present invention, the preferred methods and materials are described.

### Example 1

### Detection of Bacteria in the Air

In a first set of experiments, a device was built according to the model shown in FIGS. 1 and 2. A total number of 10⁴ E. coli K12 bacteria (in 100 μL) was manually added to the filter which was next installed on the drive shaft between the two (2) filter holders. The phage concentration of the detection fluid was adjusted to 10⁵ pfu/mL. The filter device was next mechanically rotated (6 rpm). After a 24 hour period of incubation at room temperature (23° C.±2° C.), the detection fluid was collected from the device and assayed for the presence of phages using a standard plaque assay procedure (Balows, A, 1991, Manual of Clinical Microbiology, 5^{th} Edition; Balows, A, Hausler W J, et al, eds, American Society for Microbiology, pub; 1217 pages). The results showed a thousand-fold increase in phage concentration in the sample (see Table 1). These results show that the “rolling circle” principle can be used for the capture of bacteria and further detection using specific phages.

TABLE 1
Detection of E. coli K12 by coliphage K12
using the “rolling circle” method.

| [E. coli K12] | [Coliphage K12] (pfu/mL; −log₁₀) | |
|---|---|---|
| (cfu/mL) | | T = 24 h |

| −log10 | T = 0 | Mean | SD |
|---|---|---|---|
| 0 | 5 | 5.1 | 0.3 |
| 4 | 0 | 0 | 0 |
| 4 | 5 | 8.3 | 1.2 |

### Example 2

### Stability of Detection

In another set of experiments, the same conditions as those described in Example 1 were reproduced, except that the period of incubation was extended to 12 days. The volume of detection fluid was also measured at 0 and 12 days. The results showed a 10% decrease of volume (from 36 to 32.5 mL). These results demonstrate that the device illustrated in FIG. 1 prevents the evaporation of the reaction medium and can be used for the continuous detection of bacteria using specific phages.

### Example 3

### Detection of Bacteria in a Gaseous Environment

In another set of experiments, the device described in FIG. 1 was inserted in a plastic recipient equipped with a nebulizer. This nebulizer allows the addition of bacteria in the air of the recipient. An amount of 10⁴ E. coli K12 bacteria was “nebulized” into the chamber. The filter device was next mechanically rotated (6 rpm; 4 days, 23° C.±2° C.). Samples were collected 3 and 4 days after the addition of bacteria. The results showed a million-fold increase in phage concentration of the sample (see Table 2). These results demonstrate that the “rolling circle” or “spinning wheel” principle can be used for the capture of bacteria in the air and further detection using specific phages.

TABLE 2
Detection of E. coli K12 in the air by phage K12.

| Time (days) | [Phage] (pfu/mL; −log₁₀) |
|---|---|
| 0 | 4 |
| 3 | 9.8 |
| 4 | 9.3 |

### Example 4

### Specific Uses

The present invention may be used for different purposes. Specific examples include:

a) Detection of Living Bacteria in the Air

The invention described herein could be used for detection of living bacteria such as in the air. The device could be installed in a ventilation duct and switched on for a period of several days or weeks. The bacteria would be captured by the rotating filter and next detected in the detection fluid. If bacteria are alive, replication of phages in the fluid will occur, causing a 2 to 6-log increase in the phage concentration of the detection fluid. Biosensor coated with antibodies specific to the phage (anti-phage antibodies) would detect that increase.

b) Detection of Living Bacteria in Water

A device as described herein could be used for detecting living bacteria in water environments such as tap water, water from storage tanks, cooling towers, water heaters and water hoses, wastewater, rinse water, sewage water, reconditioned wastewater, septic tank sludge, swimming pools, seawater, body of water (lakes, rivers) and natural sources of water (wells, boreholes, springs). It can be used with other liquid environments such as milk, fruit juices, apple cider, wines, beers and alcohols. Such a device would be connected for example to a tap water line. The bacteria would be captured by the filter and next detected in the detection fluid when infected by the specific phages.

c) Immunodetection of Bacteria in Gaseous or Liquid Environments

The invention described herein could be used for the detection of total bacteria (living and dead) using biosensors coated with specific antibodies directed against the bacteria.

d) Immunodetection of Fungi in Gaseous or Liquid Environments

The embodiments described herein could be used for the detection of fungi, such as Aspergillus species (spp) using appropriate filters and pre-filters and biosensors coated with specific antibodies directed against the fungus.

e) Immunodetection of Protozoa in Gaseous or Liquid Environments

The invention described herein could be used for the detection of protozoa such as Giardia lamblia using appropriate filters and pre-filters and biosensors coated with specific antibodies directed against the protozoa.

f) Immunodetection of Viruses

The invention described herein could be used for the detection of viruses such as the influenza virus (orhtomyxovirus) using appropriate filters and pre-filters and biosensors coated with specific antibodies directed against the virus.

g) Immunodetection of Spores

The invention described herein could be used for the detection of spores such as Bacillus anthracis spores using appropriate filters and pre-filters and biosensors coated with specific antibodies directed against the spore. Alternatively, substances favoring the germination of spores can be added to the detection fluid. The germinated bacteria and fungi are next detected using methods in Examples 4a) to 4d).

h) Immunodetection of Toxins

The invention described herein could be used for the detection of toxins such as the Staphylococcus enterotoxin B (SEB) using appropriate filters and pre-filters and biosensors coated with specific antibodies directed against the toxin.

i) Immunodetection of Allergens

The embodiments described herein can be used for the detection of allergens such as pollens (e.g. grass pollens), fungal spores (e.g. Aspergillus), dust (e.g. microscopic mites, insect waste products) and the like using appropriate filters and pre-filters and biosensors coated with specific antibodies directed to the allergen.

i) Detection of Particles in the Air

The invention described herein could be used for the detection of particles of various size using appropriate filters and pre-filters and appropriate methods of counting. More particularly, it could be used for the detection of microorganisms in air ducts, ventilation systems, air purifiers, air conditioners and vacuum cleaners, the detection of microorganisms in cooling towers, isolation rooms, pharmaceutical and medical clean rooms, etc.

The invention described herein could also be used for the detection of living pathogens affecting patients, the device being directly connected to the mask of a ventilator for example.

As may now be appreciated, the present invention is a substantial improvement over the prior art in that microorganisms captured on the filter are then transferred to a detection fluid. The collection unit may be readily fabricated in a sufficiently small size to enable it to be used in air ducts. Several different embodiments of the invention, as described herein, include a collection unit for detection of microorganisms in gaseous fluids, a collection unit for detection of microorganisms in liquid fluids, a collection unit using a motor-driven rotating shaft for the transfer of microorganisms from the filter to the detection fluid and a collection unit using a recirculating liquid pump for the transfer of microorganisms to the detection fluid.

Although the present invention was primarily designed for detecting microorganisms in an air sample taken from an air duct of a ventilation system, it may be used for detecting microorganisms in air sample taken from various other environments, whether gaseous or liquid, as will be easily understood by reading the following description and as apparent to a person skilled in the art. For this reason, the expressions “air”, “duct”, “ventilation”, “air-monitoring unit” and the like should not be taken as to limit the scope of the present invention and include all other kinds of substances with which the present invention may be used and could be useful.

Moreover, in the context of the present invention, the expressions “liquid” and “fluid”, as well as any other equivalent expressions and/or compound words thereof, such as “detection fluid” for example, may also be used interchangeably. The same applies for any other mutually equivalent expressions, such as “unit”, “device” and “system” for example, as well as “detecting”, “monitoring”, “analysing”, “examining” and the like, as apparent to a person skilled in the art.

In addition, although the preferred embodiment of the present invention as illustrated in the accompanying drawings comprises various components such as valves, pre-filter, post-filter, pumps, blower, container, control board, motor, lines, etc., and although the preferred embodiments of the detecting device/system and corresponding parts of the present invention as shown consist of certain geometrical configurations as explained and illustrated herein, not all of these components and geometries are essential to the invention and thus should not be taken in their restrictive sense, i.e. should not be taken as to limit the scope of the present invention. It is to be understood, as also apparent to a person skilled in the art, that other suitable components and cooperations thereinbetween, as well as other suitable geometrical configurations may be used for the detecting device/system according to the present invention, as will be briefly explained hereinafter, without departing from the scope of the invention.

While several embodiments of the invention have been described herein, it will be understood that the present invention is capable of further modifications, and this application is intended to cover any variations, uses, or adaptations of the invention, following in general the principles of the invention and including such departures from the present disclosure as to come within knowledge or customary practice in the art to which the invention pertains, and as may be applied to the essential features hereinbefore set forth and falling within the scope of the invention as defined in the appended claims.

1. A method for monitoring and detecting the presence of microorganisms in an air sample taken from an air stream, the method comprising the steps of:
a) circulating the air stream through an upper chamber, the upper chamber having an inlet for receiving the air stream into the chamber and an outlet for releasing the air stream from the chamber, the air sample taken from the air stream circulating through the upper chamber from the inlet to the outlet thereof;
b) defining a lower chamber, separated from the upper chamber, via a separator plate, the lower chamber containing liquid receiving means;
c) capturing with a filter microorganisms from said air sample, the filter extending within both the upper and lower chambers through a slot of the separator plate, the filter having a portion positioned within the upper chamber between the inlet and the outlet thereof for allowing the air sample to pass through said portion while capturing microorganisms from said air sample;
d) recovering from the filter with a liquid at least some microorganisms captured by the filter in step c), and conveying said at least some microorganisms captured into the liquid receiving means via the liquid; and
e) detecting the presence of said at least some microorganisms in liquid present in the liquid receiving means.

2. The method of claim 1, wherein the circulating step comprises the step of continuously circulating the air stream through the upper chamber.

3. The method of claim 1, wherein the detecting step is carried out with a biosensor.

4. The method of claim 1, wherein the detecting step is carried out with a biosensor comprising antibodies for specifically recognizing said at least some microorganisms.

5. The method of claim 1, wherein the detecting step is carried out with a biosensor comprising antibodies for specifically recognizing bacteriophages capable of replicating in said at least some microorganisms.

6. The method of claim 5, wherein the recovering step is carried out with a liquid comprising a culture medium comprising bacteriophages.

7. The method of claim 1, wherein the air sample is taken from an air stream provided from a ventilation duct.

8. The method of claim 1, wherein the air sample consists of an air mixture effervesced from a liquid mixture.

9. A device for monitoring and detecting the presence of microorganisms in an air sample taken from an air stream, the device comprising:
an upper chamber through which said air stream is circulated, the upper chamber having an inlet for receiving the air stream into the chamber and an outlet for releasing the air stream from the chamber, the air sample taken from the air stream circulating through the upper chamber from the inlet to the outlet thereof;
a lower chamber, separated from the upper chamber via a separator plate;
a filter extending within both the upper and lower chambers through a slot of the separator plate, the filter having a portion positioned within the upper chamber between the inlet and the outlet thereof for allowing the air sample to pass through said portion while capturing microorganisms from said air sample;
liquid receiving means positioned within the lower chamber for receiving a liquid, the liquid receiving means and the filter cooperating with each other for ensuring a contact of said portion of the filter with said liquid and thereby recovering at least some microorganisms captured by the filter and conveying them into the liquid receiving means via the liquid; and
detecting means positioned in the lower chamber for detecting the presence of said at least some microorganisms within liquid present in the liquid receiving means.

10. The device of claim 9, wherein the air stream is continuously circulated through the upper chamber.

11. The device of claim 9, wherein the detecting means comprise a biosensor.

12. The device of claim 11, wherein the biosensor comprises antibodies for specifically recognizing said at least some microorganisms.

13. The device of claim 11, wherein the biosensor comprises antibodies for specifically recognizing bacteriophages capable of replicating in said at least some microorganisms.

12. The device of claim 9, wherein the detecting means comprise an immunochromatographic detector.

13. The device of claim 9, wherein the filter consists of an assembly having a filtering membrane positioned between a pair of perforated holders, the filtering membrane being rotatable within the upper and lower chambers and the filtering membrane having a surface in contact with the liquid.

14. The device of claim 9, wherein the filter comprises a filtering membrane, a portion of the liquid in the chambers flowing from a top portion of the membrane toward a bottom portion thereof.

15. The device of claim 9, wherein the inlet of the upper chamber is provided with a pre-filter positioned upstream of the filter for filtering the air sample from substances having a size larger than microorganisms.

16. The device of claim 9, wherein the outlet of the upper chamber is provided with a post-filter for filtering the air sample released from the upper chamber.

17. The device of claim 9, wherein the lower chamber comprises a liquid outlet valve for allowing collection of a sample of the liquid contained in the lower chamber.
